(19) United States
(12) Patent Application Publication
(10) Pub. No.: US 2017/0049382 A1
(43) Pub. Date: Feb. 23, 2017

(54) Nonlinear Diagnostic Analysis Apparatus, and Essential Oils Evaluation Method Using the Same

(71) Applicant: Li-Yun Tu, Tainan City (TW)

(72) Inventor: Li-Yun Tu, Tainan City (TW)

(21) Appl. No.: 14/829,694

(22) Filed: Aug. 19, 2015

(51) Int. Cl. A61B 5/00

(52) U.S. Cl. CPC A61B 5/486; A61B 5/742; A61B 5/6814

(57) ABSTRACT

A nonlinear diagnostic analysis apparatus includes at least one transducer connected to an examinee's head for transmitting a transmission frequency and feeding back a reflection frequency from the examinee's head through the transducer, a detection unit connected to the transducer to allow the transducer to transmit the transmission frequency and receive the reflection frequency, and a control unit connected to the detection unit. The control unit has a computing unit for computing the reflection frequency and comparing the transmission frequency and reflection frequency to obtain detected information, an essential oils database containing essential oils information and essential oils using methods, such that after the computing unit computes the reflection frequency, the detected information and essential oils database are compared to select suitable type and dosage of the essential oil, and a screen for displaying the detected information and the suggested type and using method of the essential oil.

### BACKGROUND OF THE INVENTION

Field of the Invention

This disclosure relates to a nonlinear diagnostic analysis apparatus and an essential oils evaluation method using the apparatus, and more particularly to an apparatus and method that transmits a transmission frequency to a human body, such that the human body feeds back a reflection frequency and further analyzes and compares the transmission frequency and the reflection frequency to suggest the type of an essential oil to users.

Description of the Related Art

Conventional physical examination is one of the linear diagnoses used for checking whether or not a change occurs in organs, and the physical examination is provided for examinees to know about their personal health conditions, discover early symptoms or risk factors and take treatments at early stage. The examination made by conventional medical instruments may be invasive and thus causing injuries to human body. For example, these instruments may involve x-ray or nuclear magnetic resonance (NMR) radiation and their side effect cannot be eliminated easily. On the other hand, nonlinear diagnoses give diagnostic results before the occurrence of organ morphology and observe all of the changing process. The so-called nonlinear diagnosis compares the transmission and receiving frequencies to discover an abnormal condition of an examinee's organ. Compared with the conventional medical diagnosis, the nonlinear diagnosis does not examine organ morphology, but it focuses at the balance of physiological processes, so that the nonlinear diagnosis is extensively used by people. Therefore, if a diagnosis can be made before causing any organ morphology an essential oil is used at the same time, then the soothing effect can be achieved. Essential oil is an aromatic organic compound formed by photosynthesis and an aromatic factor generally extracted from plants. The essential oils generally exist in all parts of a plant including roots, trunks, barks, tree stems, leaves, and flowers. Essential oils in petals may attract beneficial insects to come close and drive harmful insects away, so that we use this property of the essential oils to drive pest away and protect our physical health. Therefore, the discloser of this disclosure conducted extensive researches and experiments and finally developed the apparatus and method in accordance with this disclosure.

### SUMMARY OF THE INVENTION

Therefore, it is a primary objective of this disclosure to provide a nonlinear diagnostic analysis apparatus and an essential oils evaluation method that transmit a transmission frequency to a human body, and the human body further feeds back a reflection frequency, and then the transmission frequency and the reflection frequency are analyzed and compared to provide suggestions to the type of essential oil used, and its dosage.

To achieve the aforementioned object, the present invention provides a nonlinear diagnostic analysis apparatus comprising at least one transducer, a detection unit, and a control unit, wherein an end of the transducer is connected to an examinee's head for transmitting a transmission frequency and further making the examinee's head to feed back a reflection frequency through the transducer, and the detection unit is connected to the transducer for driving the transducer, such that the transducer can transmit the transmission frequency and receive the reflection frequency, and the control unit is connected to the detection unit for controlling the detection unit, and the control unit comprises a computing unit, for computing the reflection frequency and comparing the transmission frequency and the reflection frequency to obtain the examinee's detected information, and the computing unit has an essential oils database with a plurality of essential oils information and essential oils using methods built therein. After the computing unit computers the reflection frequency, the detected information and the essential oils database are compared to select suitable type and dosage of the essential oil. The control unit has a screen for displaying the detected information and the suggested type and using method of the essential oil.

In the aforementioned nonlinear diagnostic analysis apparatus, the detected information includes the structure of all organs of the examinee and detected data.

In the aforementioned nonlinear diagnostic analysis apparatus, the control unit is coupled to at least one display device provided for the examinee's viewing.

In the aforementioned nonlinear diagnostic analysis apparatus, the control unit is a notebook computer.

In the aforementioned nonlinear diagnostic analysis apparatus, the control unit is a computer host.

Another objective of this disclosure is to provide an essential oils evaluation method comprising the steps of: connecting an end of at least one transducer to an examinee's head and the other end of the transducer to a detection unit, and the detection unit driving the transducer to transmit the transmission frequency and receive the reflection frequency; and connecting a control unit with the detection unit to control the detection unit; the control unit comprising: a computing unit, for computing the reflection frequency and comparing the transmission frequency and the reflection frequency to obtain the examinee's detected information; an essential oils database, having a plurality of essential oils information and essential oils using methods built therein; thereby after the computing unit computes the reflection frequency, the detected information and the essential oils database are compared to select suitable type and dosage of the essential oil; and a screen, for displaying the detected information, and the suggested type and using method of the essential oil.

After the computing unit computes the reflection frequency, the reflection frequency and the transmission frequency are compared to obtain the examinee's detected information, and the detected information is compared with the essential oils database to select the suitable type and dosage of the essential oil, and the selected type, dosage, and using method of the essential oil are displayed on a screen for the examinee's reference.

### BRIEF DESCRIPTION OF THE DRAWINGS

FIG. 1 is a schematic block diagram of a preferred embodiment of this disclosure; and

FIG. 2 is a flow chart of an essential oils evaluation method of a preferred embodiment of this disclosure.

### DETAILED DESCRIPTION OF THE PREFERRED EMBODIMENTS

The technical characteristics, contents, advantages and effects of the present invention will be apparent with the detailed description of a preferred embodiment accompanied with related drawings as follows.

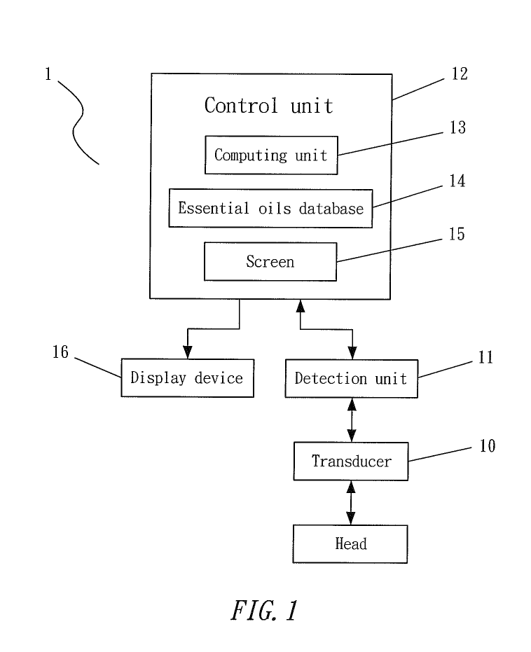

With reference to FIG. 1 for a nonlinear diagnostic analysis apparatus in accordance with this disclosure, the nonlinear diagnostic analysis apparatus 1 comprises:
at least one transducer 10, with an end connected to an examinee's head for transmitting a transmission frequency, and further making the examinee's head to feed back to a reflection frequency through the transducer 10;
a detection unit 11, connected to the transducer 10, for driving the transducer 10 to transmit the transmission frequency and receive the reflection frequency; and
a control unit 12, such as a notebook computer or a computer host, connected to the detection unit 11, for controlling the detection unit 11, and the control unit 12 comprising: a computing unit 13, for computing the reflection frequency, and comparing the transmission frequency and the reflection frequency to obtain the examinee's detected information; an essential oils database 14, having a plurality of essential oils information and essential oils using methods built therein; and a screen 15, for displaying the detected information, and the suggested type and using method of the essential oil.

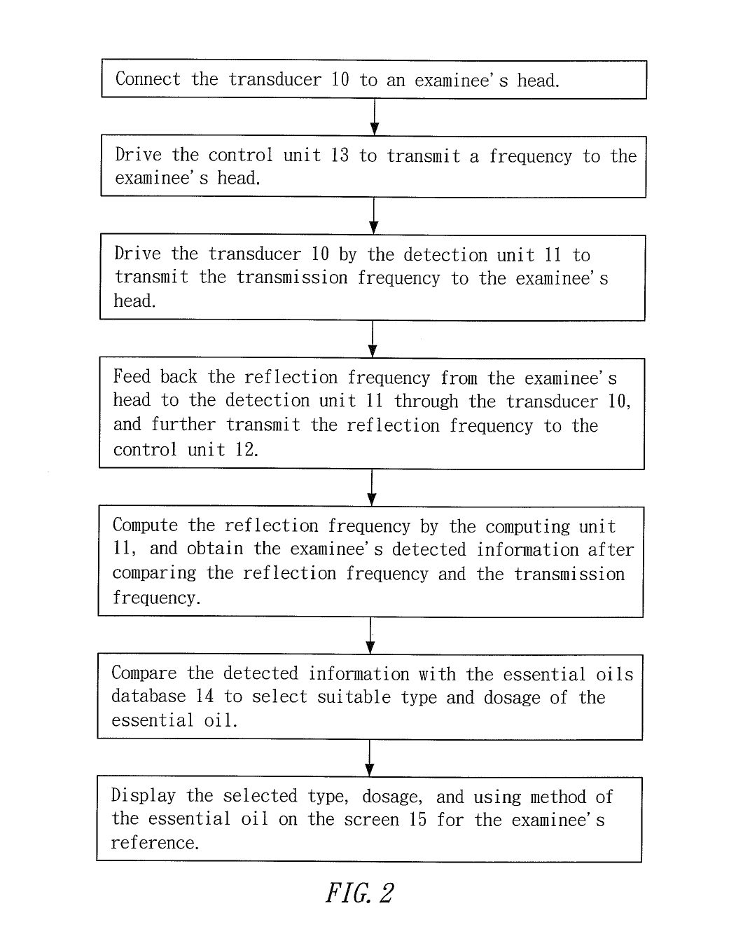

With reference to FIGS. 1 and 2 for the application of the nonlinear diagnostic analysis apparatus, the transducer 10 is connected to an examinee's head, so that the control unit 12 may transmit a control signal to the detection unit 11, and the detection unit 11 drives the transducer 10 to transmit the transmission frequency to the examinee's head and make the examinee's head to feed back the reflection frequency to the detection unit 11 through the transducer 10 and further to the control unit 12. The computing unit 11 computes the reflection frequency. After the reflection frequency and the transmission frequency are compared, the examinee's detected information is obtained, and the detected information and the essential oils database 14 are compared to select the suitable type and dosage of the essential oil, and the selected type, dosage, and using method of the essential oil are displayed on the screen 15 for the examinee's reference. Alternatively, the type, dosage, and using method of the essential oil may be displayed on a display device 16 to facilitate the examinee's viewing.

In the aforementioned nonlinear diagnostic analysis apparatus 1, the detected information includes the structure of all organs of the examinee and detected data.

The essential oils evaluation method of this disclosure is used in the nonlinear diagnostic analysis apparatus 1, and the essential oils evaluation method comprises the following steps:
(1) Connect an end of at least one transducer 10 to an examinee's head and the other end of the transducer 10 to a detection unit 11, wherein the detection unit 11 drives the transducer 10 to transmit the transmission frequency and receive the reflection frequency; and
(2) Connect a control unit 12 to the detection unit 11 to control the detection unit 11, wherein the control unit 12 comprises: a computing unit 13, for computing the reflection frequency, and comparing the transmission frequency and the reflection frequency to obtain the examinee's detected information; an essential oils database 14, having a plurality of essential oils information and essential oils using methods built therein; thereby after the computing unit 13 computes the reflection frequency, the detected information and the essential oils database 14 are compared to select suitable type and dosage of the essential oil type and dosage; and a screen 15, for displaying the detected information and the suggested type and using method of the essential oil.

Therefore, this disclosure has the following advantages and effects:
1. The analysis of the examinee's organ causes no damages to human body.
2. The use of the essential oils causes no damages to human body.

In summation of the description above, this disclosure complies with the patent application requirements, and thus is duly filed for patent application. While the disclosure has been described by means of specific embodiments, numerous modifications and variations could be made thereto by those skilled in the art without departing from the scope and spirit of the disclosure set forth in the claims.

What is claimed is:

1. A nonlinear diagnostic analysis apparatus, comprising:
at least one transducer, with an end connected to an examinee's head, and transmitting a transmission frequency, and further making the examinee's head to feed back a reflection frequency through the transducer;
a detection unit, connected to the transducer, for driving the transducer to transmit the transmission frequency and receive the reflection frequency; and
a control unit, connected to the detection unit, for controlling the detection unit, and the control unit comprising: a computing unit, for computing the reflection frequency and comparing the transmission frequency and the reflection frequency to obtain the examinee's detected information; an essential oils database having a plurality of essential oils information and essential oils using methods built therein, thereby after the computing unit computes the reflection frequency, the detected information and the essential oils database are compared to select suitable type and dosage of the essential; a screen, for displaying the detected information, and the suggested type and using method of the essential oil.

2. The nonlinear diagnostic analysis apparatus according to claim 1, wherein the detected information include the structure of all organs of the examinee and detected data.

3. The nonlinear diagnostic analysis apparatus according to claim 1, wherein the control unit is coupled to at least one display device provided for the examinee's viewing.

4. The nonlinear diagnostic analysis apparatus according to claim 2, wherein the control unit is coupled to at least one display device provided for the examinee's viewing.

5. The nonlinear diagnostic analysis apparatus according to claim 1, wherein, the control unit is a notebook computer.

6. The nonlinear diagnostic analysis apparatus according to claim 1, wherein, the control unit is a computer host.

7. An essential oils evaluation method, comprising:
(1) connecting an end of at least one transducer to an examinee's head and the other end of the transducer to a detection unit, and the detection unit driving the transducer to transmit the transmission frequency and receive the reflection frequency;
(2) connecting a control unit with the detection unit to control the detection unit; the control unit comprising: a computing unit, for computing the reflection frequency and comparing the transmission frequency and the reflection frequency to obtain the examinee's detected information; an essential oils database, having a plurality of essential oils information and essential oils using methods built therein; thereby after the computing unit computes the reflection frequency, the detected information and the essential oils database are compared to select suitable type and dosage of the essential oil; and a screen, for displaying the detected information, and the suggested type and using method of the essential oil.

8. The essential oils evaluation method according to claim 7, wherein the detected information include the structure of all organs of the examinee and detected data.

9. The essential oils evaluation method according to claim 7, wherein the control unit is coupled to at least one display device provided for the examinee's viewing.

10. The essential oils evaluation method according to claim 8, wherein the control unit is coupled to at least one display device provided for the examinee's viewing.
